(19) United States
(12) Patent Application Publication
(10) Pub. No.: US 2023/0405278 A1
(43) Pub. Date: Dec. 21, 2023

(54) Multi-Directional Catheter Guard

(71) Applicant: Catheter Guardian, LLC, Spokane, WA (US)

(72) Inventor: Caleb Hollis Whitmore, Akron, OH (US)

(73) Assignee: Catheter Guardian, LLC, Spokane, WA (US)

(21) Appl. No.: 18/334,077

(22) Filed: Jun. 13, 2023

Related U.S. Application Data
(60) Provisional application No. 63/352,096, filed on Jun. 14, 2022.

(51) Int. Cl. A61M 25/02

(52) U.S. Cl. CPC A61M 25/02; A61M 2025/0213

(57) ABSTRACT

In aspects of a multi-directional catheter guard, a first end is configured for fitment with an anatomically male patient, and a second end is configured for fitment with an anatomically female patient. The multi-directional catheter guard can include an expandable enclosure that expands and closes for fitment, encompassing a catheter tubing in a direction corresponding to patient anatomy. The multi-directional catheter guard may also include a friction material that resists lateral movement of the multi-directional catheter guard from an attached position on the catheter tubing.

### RELATED APPLICATION

This application claims priority to U.S. Provisional Patent Application Ser. No. 63/352,096 filed Jun. 14, 2022 entitled “Multi-Directional Catheter Guard”, the disclosure of which is incorporated by reference herein in its entirety.

### BACKGROUND

Generally, a catheter is a flexible tube inserted through a narrow opening into a body cavity for removing fluid, such as a urinary catheter used to alleviate fluid buildup in the bladder. A catheter is also commonly used perioperative for select surgeries to assist with healing open wounds in incontinent patients, for end-of-life care, for critically ill patients, for measuring fluids in and out of a patient, and/or for any number of other in-patient and out-patient care conditions. A catheter may be temporarily used for a short period of time, or for a longer duration, such as for a patient in intensive care, in a long-term care facility, or in a nursing home.

Although useful for many patient care conditions, catheters are not free of potential problems and risk to a patient. Catheter associated urinary tract infections (CAUTIs) are the most commonly acquired infection (over 40%) for patients while hospitalized according to the Center for Disease Control. It is estimated that CAUTIs cause approximately 9,000 preventable deaths every year in the United States, and over 500,000 yearly infections in patients who are in hospital settings. The costs and expenses associated with these types of infections are estimated to range from $10-20 billion dollars annually for bedside-care healthcare organizations. Nearly all urinary tract infections present in the form of bacteria that creeps along the outside edge of a catheter tube, and enters the bladder of a patient. Solutions to decrease the infection rates associated with catheter use are urgently needed, primarily to save lives. While not using a catheter is the most ideal and common professional practice, ultimately, one out of four patients admitted to a hospital continue to require catheters during their hospital stay.

### BRIEF DESCRIPTION OF THE DRAWINGS

Implementations of the techniques for multi-directional catheter guard are described with reference to the following Figures. The same numbers may be used throughout to reference like features and components shown in the Figures:

FIG. 1 illustrates an example of a multi-directional catheter guard in accordance with one or more implementations as described herein.

FIGS. 2-6 illustrate examples of additional aspects and features of the multi-directional catheter guard in accordance with one or more implementations as described herein.

FIG. 7 illustrates example method(s) of a multi-directional catheter guard in accordance with one or more implementations of the techniques described herein.

### DETAILED DESCRIPTION

Implementations of a multi-directional catheter guard are described, and provide techniques to solve healthcare-acquired urinary tract infections. As noted above, catheter associated urinary tract infections (CAUTIs) cause approximately 9,000 preventable deaths every year in the United States, with costs and expenses associated with these types of infections estimated to range from $10-20 billion dollars annually for healthcare organizations. Notably, the care for a patient that acquires a CAUTI is not covered by medical insurance due to the infection being deemed a hospital or care provider's fault while the patient is under care. Further, punitive costs and actions may be levied by regulatory agencies against hospitals and/or health care providers for preventable deaths attributable to a CAUTI. In addition to the preventable loss of life, these punitive costs can be substantial, also estimated in the millions of dollars yearly. Although the extent of lives lost and the associated costs and expenses are both excessive, there are currently no viable options or solutions effectively solving the problem of harmful bacteria entering a patient via the outer edge of a catheter.

Aspects of a multi-directional catheter guard (also referred to herein as a “catheter guard”) provide an antibacterial solution application device to solve the preventable healthcare-acquired urinary tract infections, thus saving lives and potentially billions of dollars in associated healthcare costs. In one or more implementations, a multi-directional catheter guard (also referred to as a “CAUTI patch”) is a physical, cylinder-shaped foam applicator, which snaps onto the outer circumference and/or edge of a catheter tubing, and maintains a physical and chemical barrier to human waste and bacteria, as well as prevents unnecessary sliding or traction movement of the standard urinary catheter as the patient naturally moves around during a hospital stay.

A multi-directional catheter guard can be implemented as disposable for hand-held, one-time use, and may be packaged (e.g., in a clear package) with a packet of antibacterial solution inside the packaging. A healthcare professional can then grab the package with one hand and squeeze to rupture the antibacterial solution packet, thus saturating the high-density, open-cell foam of the multi-directional catheter guard. The healthcare professional can then open the package with the soaked CAUTI patch device, and while holding a catheter tubing with one hand, simply pop the catheter guard onto the catheter tubing and slide the catheter guard into position for the patient.

In aspects of the disclosure, a multi-directional catheter guard has a first end for fitment with an anatomically male patient, and has a second end for fitment with an anatomically female patient. The catheter guard can be used with an antibacterial solution to prevent infection. The catheter guard has an expandable enclosure to encompass a catheter tubing, and can include a locking groove to lock onto the catheter tubing. Additionally, the catheter guard may be implemented with a spiraling rigidity support to maintain a shape of the device. The multi-directional catheter guard can also be implemented with a friction material to resist sliding on the catheter tubing, such as grip bands, non-slip dots, or any other type of resistive material that resists sliding on a catheter tubing.

In other aspects of the disclosure, a multi-directional catheter guard attaches to a catheter tubing in a direction corresponding to patient anatomy, and resists lateral movement from an attached position on the catheter tubing. The catheter guard locks onto the catheter tubing with a locking groove and/or maintains a shape of the multi-directional catheter guard with a spiraling rigidity support. The catheter guard prevents infection with an antibacterial saturating the multi-directional catheter guard.

In other aspects of the disclosure, a multi-directional catheter guard has an expandable enclosure to expand and close for fitment encompassing a catheter tubing in a direction corresponding to patient anatomy. The catheter guard can also include a locking groove to lock the multi-directional catheter guard onto the catheter tubing. In one or more implementations, the catheter guard can include an antibacterial that saturates the multi-directional catheter guard preventing infection. The catheter guard can have a spiraling rigidity support to maintain a shape of the multi-directional catheter guard. The catheter guard can also include a friction material to resist lateral movement (e.g., sliding) of the multi-directional catheter guard from an attached position on the catheter tubing. The friction material may include grip bands extending about an internal circumference of the multi-directional catheter guard to resist the lateral movement. Alternatively or in addition, the friction material may include non-slip dots contacting the catheter tubing to resist the lateral movement.

While features and concepts of multi-directional catheter guard can be implemented in any number of different environments and/or configurations, implementations of multi-directional catheter guard are described in the context of the following example descriptions and methods.

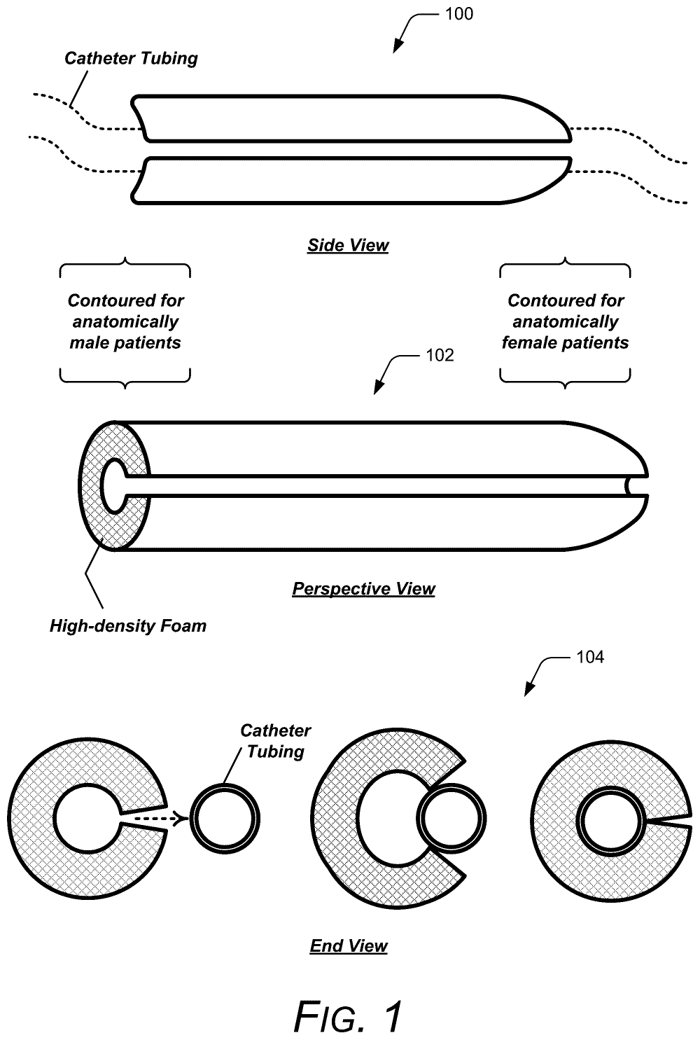

FIG. 1 illustrates an example 100 of a multi-directional catheter guard, as described herein. In this example 100, the multi-directional catheter guard is shown in a side view encompassing a catheter tubing. As illustrated, the multi-directional catheter guard has a first end for fitment with an anatomically male patient, and has a second end for fitment with an anatomically female patient. A perspective view at 102 also illustrates a configuration of the multi-directional catheter guard implemented with a high-density, open-cell foam that can soak up and become saturated with an antibacterial solution. An end view at 104 also illustrates the multi-directional catheter guard attaching to a catheter tubing.

In an implementation, the multi-directional catheter guard is a moisturized or liquid applicator, which may be approximately 4.5 inches long and 1.1 inches in diameter (however, any size catheter guard may be implemented). The multi-directional catheter guard can be convex-cone shaped on one end, and have a concave shape on the other end. Notably, a multi-directional catheter guard may be formed as any of various shapes and/or tapered on one or both ends to accommodate patient applications. The multi-directional catheter guard has a lengthwise opening along the entire guard and through the foam to the center diameter of the cylinder shape, which opens enabling the catheter guard to squeeze onto and conform to the outside of a catheter tubing. The size of the circular hollow center of the multi-directional catheter guard can be formed to accommodate any size of catheter tubing (approximately 0.21″ in an implementation). A catheter tubing is typically a flexible rubber tubing and the multi-directional catheter guard can be sized to hold in place over the catheter tubing.

In one or more implementations, the lengthwise opening through the catheter guard to the internal, circular hollow center (or central lumen) is uniquely designed to both pop onto and off of the catheter tubing, yet also attach firmly enough to remain steady and in position. The end view of the multi-directional catheter guard at 104 illustrates an example shape of the lengthwise opening through the foam to the center diameter of the cylinder shape of the catheter guard.

In this example, the lengthwise opening is a narrow wedge shape, or V-shape, or Y-shape, that narrows to only a slit at the inner center diameter of the circular hollow center of the catheter guard, as viewed longitudinally. This allows a user to push the catheter guard onto the catheter tubing with one hand, yet the catheter guard closes enough to keep physical contaminants (e.g., bacteria and human waste) from entering through the lengthwise opening onto the catheter tubing. Further, although the side view at 100 and the perspective view 102 of the multi-directional catheter guard illustrate the lengthwise opening through the foam of the catheter guard as having a space between the longitudinal edges of the opening, in practice, the longitudinal edges of the lengthwise opening may come together (e.g., touching) when the multi-directional catheter guard is installed over and attached to the catheter tubing.

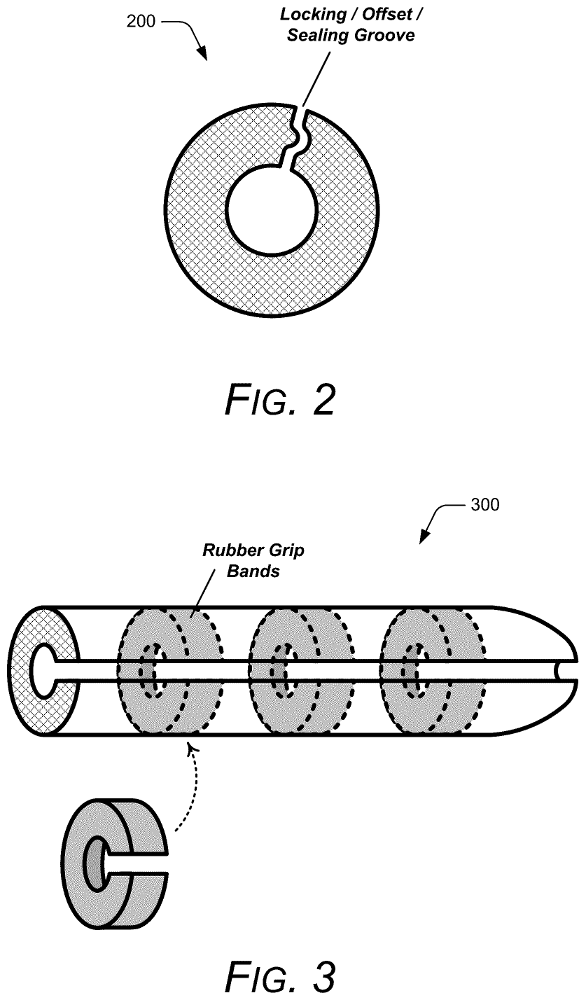

FIG. 2 illustrates an example 200 of an alternative configuration of the lengthwise opening through the foam to the center diameter of the cylinder shape of the multi-directional catheter guard. In this example 200, the lengthwise opening through the catheter guard is a locking, offset, and/or sealing groove with a key-lock design cut that locks the catheter guard onto the catheter tubing. One side of the lengthwise opening fits into the other, like a dove-tail or any other interlocking shape formed into the foam of the catheter guard to lock the catheter guard onto the catheter tubing. The interlocking shape of the locking groove lengthwise opening through the catheter guard also provides an additional barrier to physical contaminates that may cause an infection in a patient. Although shown and described as a “locking groove” (which may also be an offset groove), the lengthwise opening in the multi-directional catheter guard may be implemented as any type of groove, opening, offset, etc. Notably, the lengthwise opening of the multi-directional catheter guard is not limited by the example designs shown and described herein, but rather may be implemented as any shape, opening, and/or enclosure.

As an alternative to the parallel, longitudinal lengthwise opening shown in the illustrations of the multi-directional catheter guard, a diagonal lengthwise opening may be implemented that starts lower on one end and runs in a direction diagonally to the other end, creating a slight twist in the opening. This configuration of a diagonal lengthwise opening would make it more difficult for the catheter guard to accidently pop off of the catheter tubing with a unidirectional force, and would require intentional, multidirectional force from a person to remove the catheter guard from the catheter tubing.

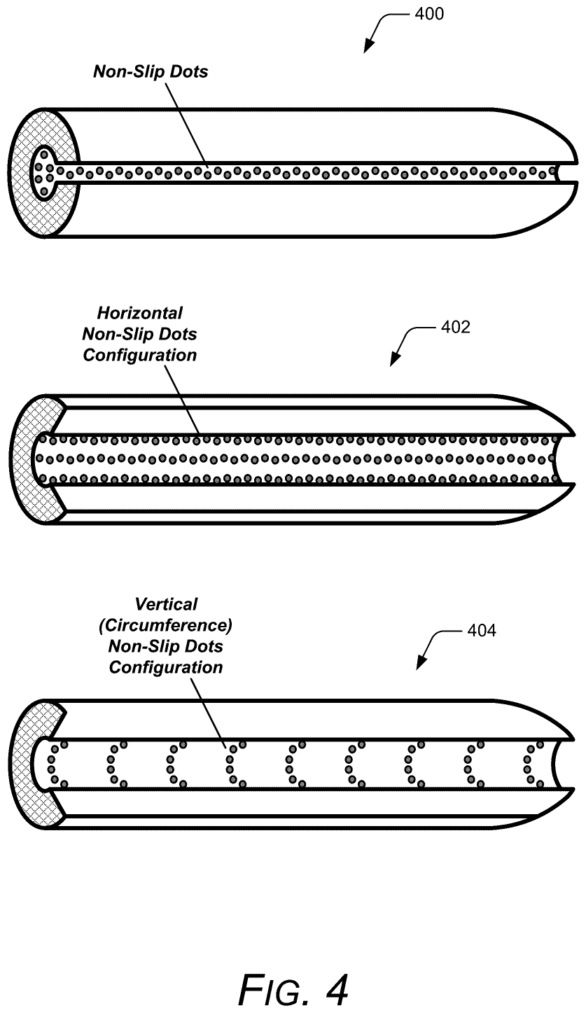

FIG. 3 illustrates an example 300 of a multi-directional catheter guard with friction material to facilitate maintaining the catheter guard in a set position on a catheter tubing. In aspects of using the multi-directional catheter guard, a healthcare professional can grip the catheter guard and move it into position for the patient by sliding it along the catheter tubing. However, once positioned in place for the patient, ideally the catheter guard would remain in-place as the patient naturally moves around, such as when lying in bed during a hospital stay. To facilitate maintaining the multi-directional catheter guard in a set position, the catheter guard may include grip bands extending about an internal circumference of the catheter guard to resist lateral movement (e.g., sliding) of the catheter guard from an attached position on the catheter tubing. The grip bands can be implemented as small rubber, silicone, or non-latex keepers spaced intermittently along the catheter guard internal lumen, which is in contact with the catheter tubing. Additionally, the grip bands each function as a mechanical barrier to prevent contaminants from longitudinally moving along the catheter tubing and possibly infecting a patient. The grip bands will keep the catheter guard snug against the patient, but also keep it from slipping down the catheter tubing. While still allowing a healthcare professional to be able to grip and slide the catheter guard along the catheter tubing, the grip bands also provide enough traction to aid in maintaining the catheter guard in place on the catheter tubing.

FIG. 4 further illustrates an example 400 of a multi-directional catheter guard with friction material to facilitate maintaining the catheter guard in a set position on a catheter tubing. In this example, non-slip dots (e.g., similar to grip dots on an oven mitt) can be implemented within the circular hollow center of the catheter guard to keep the catheter guard from sliding up or down on the outside of the catheter tubing. In implementations, one or more beads of non-slip dots may be run horizontally lengthwise down the hollow center of the catheter guard, parallel to the lengthwise opening as shown at 402. Alternatively, several beads of the non-slip dots may be arranged in vertical circumference configurations, as shown at 404 within the hollow center of the catheter guard. Alternatively, the non-slips dots can be simply arranged in a peppered configuration throughout the hollow center of the catheter guard as generally shown at 400, within the internal lumen of the catheter guard that is in contact with the catheter tubing. In one or more implementations, any type of alternative anti-slide compound, system, or configuration may be implemented in a multi-directional catheter guard to provide enough traction to aid in maintaining the catheter guard in place on the catheter tubing.

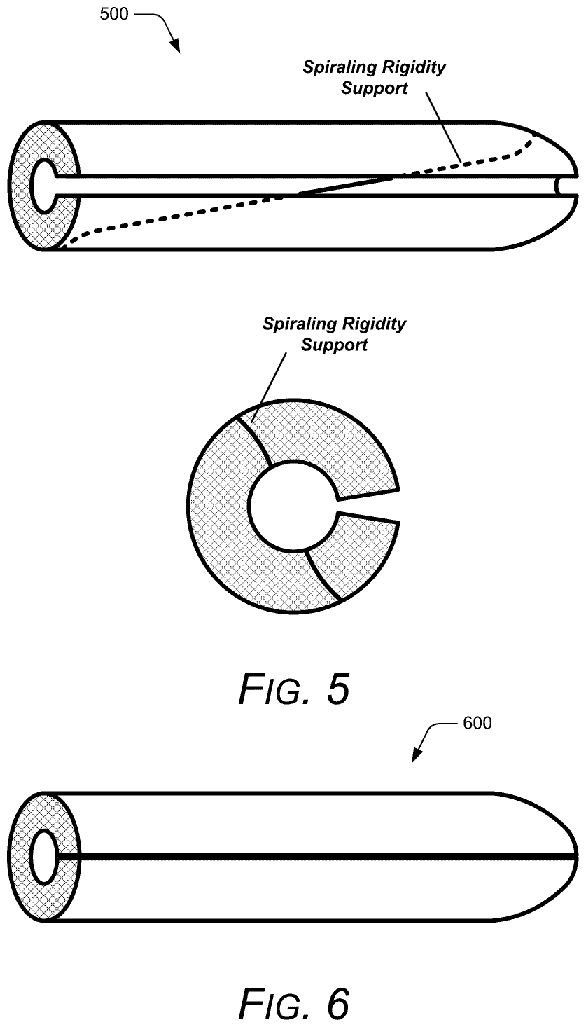

FIG. 5 illustrates an example 500 of a multi-directional catheter guard with a spiraling rigidity support that facilitates opening a parallel longitudinal lengthwise opening or a diagonal lengthwise opening of the catheter guard (e.g., such as with a tootsie-roll like action), and then after the catheter guard is placed onto the catheter tubing, the catheter guard rolls back into a diagonal clamping shape, thus securing itself in a diagonal wrapping configuration around the catheter tubing. Generally, this diagonal wrapping configuration of the multi-directional catheter guard has an elastic rigidity maintained by the spiraling rigidity support that is integrated with the catheter guard. In one or more implementations, thin vertical membranes (e.g., silicone, rubber, or any other material) may be implemented as contaminant barriers about the internal circumference of the circular hollow center (or central lumen) of the catheter guard, positioned similar as the grip bands shown in FIG. 3, where each thin vertical membrane functions as a mechanical barrier to prevent contaminants from longitudinally moving along the catheter tubing and possibly infecting a patient.

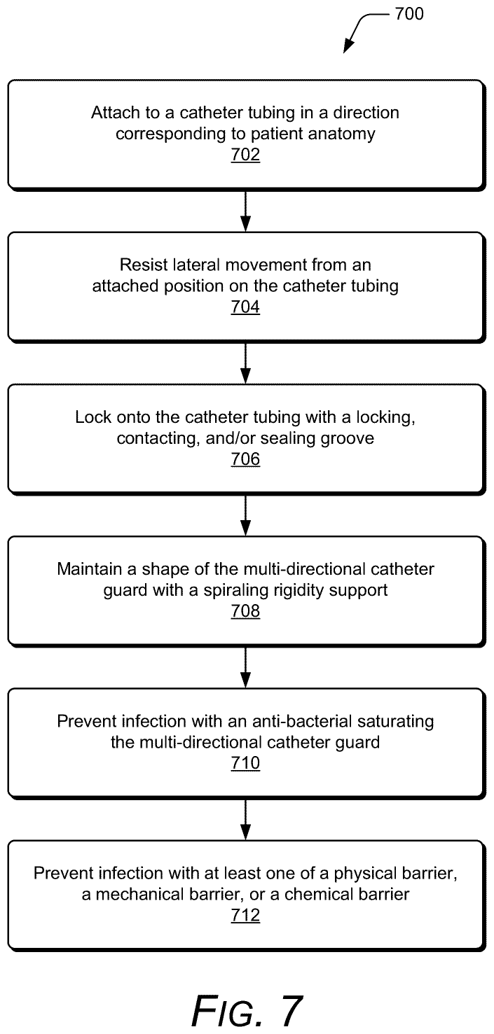

FIG. 6 further illustrates an example 600 of the multi-directional catheter guard. As described above, any one or more of a parallel, longitudinal lengthwise opening, a locking groove lengthwise opening, or a diagonal lengthwise opening through the foam of the catheter guard has enclosure edges that may fully come together (e.g., touching, contacting, sealing) when the multi-directional catheter guard is installed over and attached to the catheter tubing. The closed (e.g., touching, contacting, sealing) and sealed opening provides an additional barrier to physical contaminates that may cause an infection in a patient.

FIG. 7 illustrates example method(s) 700 of a multi-directional catheter guard. The order in which the method is described is not intended to be construed as a limitation, and any number or combination of the described method operations can be performed in any order to perform a method, or an alternate method.

At 702, a multi-directional catheter guard attaches to a catheter tubing in a direction corresponding to patient anatomy. For example, the multi-directional catheter guard has a first end for fitment with an anatomically male patient, and has a second end for fitment with an anatomically female patient.

At 704, lateral movement of the multi-directional catheter guard is resisted from an attached position on the catheter tubing. The lateral movement (e.g., sliding) may be resisted utilizing grip bands extending about an internal circumference of the multi-directional catheter guard. Similarly, the lateral movement may be resisted utilizing non-slip dots contacting the catheter tubing.

At 706, the multi-directional catheter guard locks onto the catheter tubing with a locking, contacting, and/or sealing groove. For example, the interlocking shape of a locking groove lengthwise opening through the catheter guard provides an additional barrier to physical contaminates that may cause an infection in a patient.

At 708, a shape of the multi-directional catheter guard is maintained with a spiraling rigidity support. For example, a diagonal wrapping configuration of the multi-directional catheter guard has an elastic rigidity maintained by a spiraling rigidity support integrated with the catheter guard.

At 710, infection is prevented with an antibacterial saturating the multi-directional catheter guard. For example, the multi-directional catheter guard can be implemented with a high-density, open-cell foam that soaks up and becomes saturated with an antibacterial solution.

At 712, infection is prevented with at least one of a physical barrier, a mechanical barrier, or a chemical barrier. For example, the multi-directional catheter guard can be implemented with any one or more of a physical barrier (e.g., the high-density, open-cell foam of the multi-directional catheter guard), a mechanical barrier (e.g., the grip bands or thin vertical membranes that extend about an internal circumference of the catheter guard), or a chemical barrier (e.g., the antibacterial solution).

Although implementations of a multi-directional catheter guard have been described in language specific to features and/or methods, the appended claims are not necessarily limited to the specific features or methods described. Rather, the specific features and methods are disclosed as example implementations of a multi-directional catheter guard, and other equivalent features and methods are intended to be within the scope of the appended claims. Further, various different examples are described and it is to be appreciated that each described example can be implemented independently or in connection with one or more other described examples. Additional aspects of the techniques, features, and/or methods discussed herein relate to one or more of the following:

A multi-directional catheter guard, comprising: an expandable enclosure configured to expand and close for fitment encompassing a catheter tubing in a direction corresponding to patient anatomy; and a friction material configured to resist lateral movement of the multi-directional catheter guard from an attached position on the catheter tubing.

Alternatively or in addition to the above described multi-directional catheter guard, any one or combination of: an antibacterial configured to saturate the multi-directional catheter guard preventing infection. A locking groove configured to lock the multi-directional catheter guard onto the catheter tubing. A spiraling rigidity support configured to maintain a shape of the multi-directional catheter guard. The friction material comprises grip bands extending about an internal circumference of the multi-directional catheter guard to resist the lateral movement. The friction material comprises non-slip dots contacting the catheter tubing to resist the lateral movement.

A method of a multi-directional catheter guard, comprising: attaching to a catheter tubing in a direction corresponding to patient male anatomy or female anatomy; and resisting lateral movement from an attached position on the catheter tubing.

Alternatively or in addition to the above described method of a multi-directional catheter guard, any one or combination of: the method further comprising preventing infection with an antibacterial saturating the multi-directional catheter guard. The method further comprising locking onto the catheter tubing with a locking groove. The method further comprising maintaining a shape of the multi-directional catheter guard with a spiraling rigidity support. The lateral movement is resisted based at least in part on grip bands extending about an internal circumference of the multi-directional catheter guard. The lateral movement is resisted based at least in part on non-slip dots contacting the catheter tubing.

A multi-directional catheter guard, comprising: a first end configured for fitment with an anatomically male patient; and a second end configured for the fitment with an anatomically female patient.

Alternatively or in addition to the above described multi-directional catheter guard, any one or combination of: an antibacterial configured to prevent infection. An expandable enclosure configured to encompass a catheter tubing. A friction material configured to resist sliding on a catheter tubing. A locking groove configured to lock onto a catheter tubing. A spiraling rigidity support configured to maintain a shape of the multi-directional catheter guard. Grip bands configured to resist sliding on a catheter tubing. Non-slip dots configured to resist sliding on a catheter tubing.

1. A multi-directional catheter guard, comprising:
an expandable enclosure configured to expand and close for fitment encompassing a catheter tubing in a direction corresponding to patient anatomy; and
a friction material configured to resist lateral movement of the multi-directional catheter guard from an attached position on the catheter tubing.

2. The multi-directional catheter guard of claim 1, further comprising an antibacterial configured to saturate the multi-directional catheter guard preventing infection.

3. The multi-directional catheter guard of claim 1, further comprising a locking groove configured to lock the multi-directional catheter guard onto the catheter tubing.

4. The multi-directional catheter guard of claim 1, further comprising a spiraling rigidity support configured to maintain a shape of the multi-directional catheter guard.

5. The multi-directional catheter guard of claim 1, wherein the friction material comprises grip bands extending about an internal circumference of the multi-directional catheter guard to resist the lateral movement.

6. The multi-directional catheter guard of claim 1, wherein the friction material comprises non-slip dots contacting the catheter tubing to resist the lateral movement.

7. A method of a multi-directional catheter guard, comprising:
attaching to a catheter tubing in a direction corresponding to patient male anatomy or female anatomy; and
resisting lateral movement from an attached position on the catheter tubing.

8. The method of claim 7, further comprising:
preventing infection with an antibacterial saturating the multi-directional catheter guard.

9. The method of claim 7, further comprising:
locking onto the catheter tubing with a locking groove.

10. The method of claim 7, further comprising:
maintaining a shape of the multi-directional catheter guard with a spiraling rigidity support.

11. The method of claim 7, wherein the lateral movement is resisted based at least in part on grip bands extending about an internal circumference of the multi-directional catheter guard.

12. The method of claim 7, wherein the lateral movement is resisted based at least in part on non-slip dots contacting the catheter tubing.

13. A multi-directional catheter guard, comprising:
a first end configured for fitment with an anatomically male patient; and
a second end configured for the fitment with an anatomically female patient.

14. The multi-directional catheter guard of claim 13, further comprising an antibacterial configured to prevent infection.

15. The multi-directional catheter guard of claim 13, further comprising an expandable enclosure configured to encompass a catheter tubing.

16. The multi-directional catheter guard of claim 13, further comprising a friction material configured to resist sliding on a catheter tubing.

17. The multi-directional catheter guard of claim 13, further comprising a locking groove configured to lock onto a catheter tubing.

18. The multi-directional catheter guard of claim 13, further comprising a spiraling rigidity support configured to maintain a shape of the multi-directional catheter guard.

19. The multi-directional catheter guard of claim 13, further comprising grip bands configured to resist sliding on a catheter tubing.

20. The multi-directional catheter guard of claim 13, further comprising non-slip dots configured to resist sliding on a catheter tubing.
